(19) United States
(12) Patent Application Publication
(10) Pub. No.: US 2022/0387443 A1
(43) Pub. Date: Dec. 8, 2022

(54) RESPIRATORY SYNCYTIAL VIRUS FUSION PROTEIN INHIBITOR COMPOSITIONS AND METHODS FOR THE TREATMENT AND PROPHYLAXIS OF RSV DISEASES USING THE SAME

(71) Applicant: SHANGHAI ARK BIOPHARMACEUTICAL CO., LTD., Shanghai (CN)

(72) Inventors: Cheng PENG, Shanghai (CN); Zhen Jim WU, Shanghai (CN); Yuping LI, Shanghai (CN); Chunyan ZHOU, Shanghai (CN); Yuanyuan LI, Shanghai (CN); Stephen TOOVEY, Shanghai (CN); Haiqing YUAN, Shanghai (CN)

(21) Appl. No.: 17/772,535

(22) PCT Filed: Oct. 29, 2020
(86) PCT No.: PCT/CN2020/124909

§ 371 Date: Apr. 28, 2022

Related U.S. Application Data
(60) Provisional application No. 62/929,034, filed on Oct. 31, 2019.

(51) Int. Cl. A61K 31/554; A61K 9/28; A61K 9/20

(52) U.S. Cl. CPC A61K 31/554; A61K 9/2886; A61K 9/2018; A61K 9/2009; A61K 9/2013

(57) ABSTRACT

Disclosed is a pharmaceutical unit dosage composition including a plurality of enteric coated micro pellets. Each enteric coated micro pellet includes a core bead, an optional first sealing layer, an API layer including a Compound (I) having the following structure or a pharmaceutically acceptable salt thereof, an optional second sealing layer, and an enteric coating layer. Also disclosed is a method for the treatment and prophylaxis of RSV diseases including providing a Compound (I) and administering to a patient in need thereof a therapeutically effective amount of the Compound (I) so that t½ of the Compound (I) is about 6 to 13 hours.

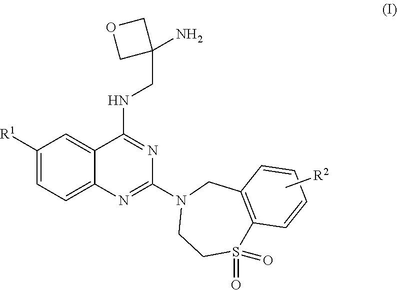

### CROSS-REFERENCE TO RELATED APPLICATION(S)

This application claims the benefit of and priority to U.S. Provisional Patent Application Ser. No. 62/929,034, filed Oct. 31, 2019, the entire disclosure of which is incorporated herein by reference.

### TECHNICAL FIELD

The present invention relates to respiratory syncytial virus fusion protein inhibitor compositions and methods for the treatment and prophylaxis of RSV diseases using the compositions. The compositions and methods described herein provides a benefit in therapeutic areas where inhibition of RSV fusion protein is desired, with minimization or elimination of adverse side effects resulting from administering such RSV fusion protein inhibitors.

### TECHNICAL BACKGROUND

Respiratory Syncytial Virus (RSV) belongs to the family of Paramyxoviridae, subfamily of Pneumovirinae. The human RSV is a major cause of acute upper and lower respiratory tract infection in infants and children. Almost all children are infected by RSV at least once by the age of two. Natural human immunity against RSV is incomplete. In normal adults and older children, RSV infection is mainly associated with upper respiratory tract symptoms. Severe cases of RSV infection often lead to bronchiolitis and pneumonia, which requires hospitalization. High-risk factors for lower respiratory tract infections include premature birth, congenital heart disease, chronic pulmonary disease, and immuno-compromised conditions. A severe infection at young age may lead to recurrent wheezing and asthma. For the elderly, RSV-related mortality rate becomes higher with advancing age.

There is no RSV vaccine available for human use, despite of many attempts in subunit vaccine and live-attenuated vaccine approaches. VIRAZOLE, the aerosol form of ribavirin, is the only approved antiviral drug for the treatment of RSV infection. However, it is rarely used clinically, due to limited efficacy and potential side effects. Two marketed prophylaxis antibodies were developed by Medimmune (CA, USA).

RSV-IGIV (brand name RespiGam) is polyclonal concentrated RSV neutralizing antibody administered through monthly infusion of 750 mg/kg in hospital (Wandstrat T. L., Ann. Pharmacother., 1997 January; 31 (1): 83-8). Subsequently, the usage of RSV-IGIV was largely replaced by palivizumab (brand name SYNAGIS), a humanized monoclonal antibody against RSV fusion (F) protein approved for prophylaxis in high-risk infants in 1998. When administered intramuscularly at 15 mg/kg once a month for the duration of RSV season, palivizumab demonstrated 45-55% reduction of hospitalization rate caused by RSV infection in selected infants (Pediatrics, 1998 September; 102 (3): 531-7; Feltes T. F., et al., J. Pediatr., 2003 October; 143 (4): 532-40). Unfortunately, palivizumab is not effective in the treatment of established RSV infection. A newer version monoclonal antibody, motavizumab, was designed as potential replacement of palivizumab, but failed to show additional benefit over palivizumab in subsequent Phase III clinical trials (Feltes T. Fetal, Pediatr. Res., 2011 Aug; 70 (2): 186-91). MEDI8897, an extended half-life respiratory syncytial virus (RSV) F monoclonal antibody (mAb) with longer half-life is currently being developed for the prevention of lower respiratory tract infection (LRTI) caused by RSV in high-risk children (ClinicalTrials.gov Identifier: NCT03959488).

A number of small molecule RSV inhibitors have been discovered. Among them, only a few reached Phase I or Phase II clinical trials. GS-5806, a potent F protein inhibitor, demonstrated efficacy in human RSV viral challenge studies by significantly reducing viral load (4.2 log 10) and disease symptom score. However, in Phase II trial in hematopoietic stem cell transplant patients, it failed to meet primary and secondary efficacy endpoints (Beigel, J. H., et al., Antiviral Research, 2019 Jul; 167). Lumicitabine (AL-8176) is an oral nucleoside analog that previously demonstrated proof of concept in a human RSV challenge model (DeVincenzo J., et al., N. Engl. J. Med., 2015; 373: 2048-2058). A single and multiple ascending dose study in infants hospitalized with RSV infection was recently completed with results showing graded treatment-emergent neutrophil abnormalities (EudraCT number: 2013-005104-33), and clinical development of this molecule was later completely abandoned. JNJ-53718678 is another small-molecule RSV fusion inhibitor that established clinical proof of concept in a Phase 2a adult RSV challenge study (Stevens, M., et al., J. Infect. Dis., 2018; 218; 748-756). Two Phase 2 studies of JNJ-53718678 in adults and infants have been initiated (ClinicalTrials.gov Identifier: NCT03379675, NCT03656510).

RNAi therapeutics against RSV have also been thoroughly studied. ALN-RSVO1 (Alnylam Pharmaceuticals, MA, USA) is a siRNA targeting on RSV gene. A nasal spray administered for two days before and for three days after RSV inoculation decreased infection rate among adult volunteers (DeVincenzo J., et al., Proc. Natl. Acad. Sci. USA, 2010 May 11; 107 (19): 8800-5). In a Phase II trial using naturally infected lung transplantation patients, results were not sufficient for conclusion of antiviral efficacy, though certain health benefits have been observed (Zamora M. R., et al., Am. J. Respir. Crit. Care Med., 2011 Feb. 15; 183 (4): 531-8). A Phase IIb clinical trials in similar patient population for ALN-RSV01 did not show significant impact on viral parameters or symptom scores, although a trend toward a decrease in new or progressive bronchiolitis obliterans syndrome was observed in certain patient cohort (Gottlieb J., et al., J. Heart Lung Transplant., 2016 Feb;35 (2): 213-21).

Therefore, safe and effective treatment for RSV disease is needed urgently.

### SUMMARY

References will now be made in details to embodiments of the present invention.

In one embodiment, the present invention discloses a pharmaceutical unit dosage composition that includes a plurality of enteric coated micro pellets. Each enteric coated micro pellet includes

a core bead;

an optional first sealing layer;

an active pharmaceutical ingredient (API) layer, the API layer including a Compound (I) having the following structure or a pharmaceutically acceptable salt thereof,

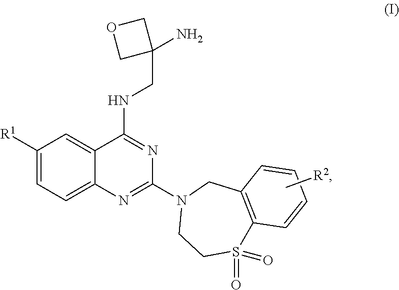

wherein
R¹ is selected from the group consisting of hydrogen, halogen, C₁-C₃ alkyl, C₁-C₃ alkoxyl, —CN, —C(O)R³, halogen substituted C₁-C₃ alkyl, and halogen substituted C₁-C₃ alkoxyl;

R² is selected from the group consisting of hydrogen, halogen, C₁-C₃ alkyl, C₁-C₃ alkoxyl, and —CN; and

R³ is selected from the group consisting of hydrogen, C₁-C₃ alkyl, and C₁-C₃ alkoxyl;

an optional second sealing layer; and

an enteric coating layer.

In another embodiment, in the structure of the Compound (I), R¹ is methyl and R² is hydrogen.

In another embodiment, the pharmaceutical unit dosage composition includes 10 to 300 mg of the Compound (I).

In another embodiment, the pharmaceutical unit dosage composition is in a form selected from the group consisting of a capsule, a tablet, and a sachet.

In another embodiment, the core bead is selected from the group consisting of a sugar sphere, also called sucrose sphere, a microcrystalline cellulose sphere, and a starch sphere, and has a diameter of 0.2 to 2 mm; and the weight of the core bead of each enteric coated micro pellet is 0.05 to 0.5 mg.

In another embodiment, the optional first sealing layer includes an adhesive agent; and the adhesive agent is selected from the group consisting of hydroxypropyl methyl cellulose, hydroxyethyl cellulose, hydroxypropyl cellulose, sodium carboxymethyl cellulose, polyvinyl alcohol, polyvinyl pyrrolidone, starch slurry, methylcellulose, and a combination thereof. It may also include talc. The weight of the optional first sealing layer of each enteric coated micro pellet is 0.001 to 0.01 mg.

In another embodiment, the optional first sealing layer also can be made by using gastric soluble film coating premix. The weight of the optional first sealing layer of each enteric coated micro pellet is 0.001 to 0.01 mg.

In another embodiment, the API layer includes the Compound (I) and an adhesive agent; and the adhesive agent is selected from the group consisting of hydroxypropyl methyl cellulose, hydroxyethyl cellulose, hydroxypropyl cellulose, polyvinyl alcohol, sodium carboxymethyl cellulose, polyvinyl pyrrolidone, starch slurry, methylcellulose, and a combination thereof. The weight of the API layer of each enteric coated micro pellet is 0.01 to 0.1 mg.

In another embodiment, the optional second sealing layer includes an adhesive agent; and the adhesive agent is selected from the group consisting of hydroxypropyl methyl cellulose, hydroxyethyl cellulose, hydroxypropyl cellulose, sodium carboxymethyl cellulose, polyvinyl alcohol, polyvinyl pyrrolidone, starch slurry, methylcellulose, ethylcellulose, and a combination thereof. It may also include talc. The weight of the second sealing layer of each enteric coated micro pellet is 0.002 to 0.02 mg.

In another embodiment, the optional second sealing layer also can be made by using gastric soluble film coating premix. Gastric soluble film coating premix includes adhesive agent, plasticizer, and colorant (e.g. OPADRY® Complete Film Coating System). The weight of the optional second sealing layer of each enteric coated micro pellet is 0.002 to 0.02 mg.

In another embodiment, the enteric coating layer includes 30-95 wt % of an enteric coating material, 1-40 wt % of a plasticizer, 1-20 wt % of an anti-caking agent, and 0.5-20 wt % of an emulsifier; the enteric coating material is selected from the group consisting of acrylic resin, hydroxypropyl methyl cellulose, hydroxypropyl methyl cellulose phthalate, cellulose acetate phthalate, polymethacrylates, methacrylic acid-ethyl acrylate copolymer, methacrylic acid copolymer, ethylene-vinyl acetate copolymer, and a combination thereof, the plasticizer is selected from the group consisting of triethyl citrate, polyethylene glycol, tributyl citrate, dibutyl sebacate, diethyl phthalate, and a combination thereof; the anti-caking agent is selected from the group consisting of glycerin monostearate and talc; the emulsifier is selected from the group consisting of Tween and sodium lauryl sulfate; and the weight of the enteric coating layer of each enteric coated micro pellet is 0.01 to 0.1 mg.

In another embodiment, the enteric coating layer includes at least one of Eudragit L30D-55, Eudragit S100, and Eudragit L100. The weight of the enteric coating layer of each enteric coated micro pellet is 0.01 to 0.1 mg.

In another embodiment, the enteric coating layer includes mixed emulsion of water resistant and plasticizer (e.g. PlasACRYL® HTP 20). The weight of the enteric coating layer of each enteric coated micro pellet is 0.01 to 0.1 mg.

In another embodiment, the pharmaceutical unit dosage composition includes a plurality of enteric coated micro pellets and an anti-sticking agent. The anti-sticking agent is selected from the group consisting of silicon dioxide, stearic acid, sodium stearyl fumarate, magnesium stearate, talc, and a combination thereof. The weight ratio of the anti-sticking agent to the enteric coated micro pellets is 0.0005-0.1:1.

In one embodiment, the particle size D₉₀ of the API in the enteric coated micro pellet is no more than 100 m.

In one embodiment, the present application also provides a method for the treatment and prophylaxis of RSV diseases. The method includes the following steps:

providing a Compound (I) having the following structure or a pharmaceutically acceptable salt thereof,

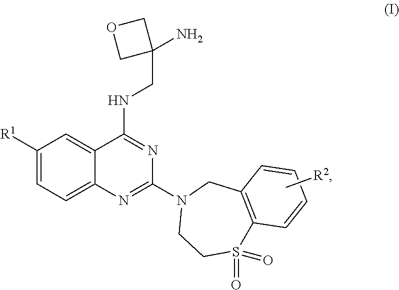

wherein

R¹ is selected from the group consisting of hydrogen, halogen, C₁-C₃ alkyl, C₁-C₃ alkoxyl, —CN, —C(O)R³, halogen substituted C₁-C₃ alkyl, and halogen substituted C₁-C₃ alkoxyl;

R² is selected from the group consisting of hydrogen, halogen, C₁-C₃ alkyl, C₁-C₃ alkoxyl, and —CN; and

R³ is selected from the group consisting of hydrogen, C₁-C₃ alkyl, and C₁-C₃ alkoxyl; and

administering to a patient in need thereof a therapeutically effective amount of the Compound (I).

In another embodiment, in the structure of the Compound (I), R¹ is methyl and R² is hydrogen.

In another embodiment, the therapeutically effective amount of the Compound (I) is 200 mg to 600 mg once per day for adult patient.

In another embodiment, the therapeutically effective amount of the Compound (I) is 100 mg to 300 mg every 12 hours for adult patient.

In another embodiment, the therapeutically effective amount of the Compound (I) is 1 mg to 10 mg per kilogram body weight once per day for pediatric patient.

In another embodiment, the therapeutically effective amount of the Compound (I) is 1 mg to 8 mg per kilogram body weight every 12 hours for pediatric patient.

In another embodiment, when administering to a patient in need thereof a therapeutically effective amount of the Compound (I), an elimination half-life (t½) of the Compound (I) is about 6 to 13 hours.

### DETAILED DESCRIPTION

The present invention is based on design and detailed experiments and clinical trials, that technical problems such as instability and poor absorption previously believed to be indicative of the 4-(((3-aminooxetan-3-yl)methyl)amino)-quinazoline derivative Compound (I) can be improved to clinically insignificant levels by the creation of a composition and a dosing regimen. This design enabled the development of a unit dosage form that incorporates Compound (I) in about 10 to about 300 mg per unit dosage form that are suitable for administration to human patients of different ages and different bodyweights, such as infants, toddlers, children, adolescents, and adults. Such unit dosage form, when orally administered, provides therapeutically useful pharmacokinetic characteristics and minimizes degradation previously believed unavoidable. The pharmacokinetic characteristics of Compound (I), such as maximum drug concentration (Cₘₐₓ), time to maximum drug concentration (Tₘₐₓ), lowest drug concentration at steady state (C_{trough}), and drug exposure (AUC), are critical in achieving desired therapeutic effects while minimizing side effects. The degradation of Compound (I) can occur in acidic environment such as in the stomach or in the gastrointestinal tract and could contribute to the side effects of the drug. The present invention of a unit dosage form such as a capsule containing about 10 to about 300 mg of the Compound (I) as enteric coated micro pellets or dry powder for suspension containing about 0.01 to 0.60 g of the Compound (I) and the dosing regimen of once or twice daily up to a maximum of 600 mg Compound (I) per day allows the administration of Compound (I) to patients suffering from RSV infection, including pediatric patients.

As described herein, the Compound (I), or a pharmaceutically acceptable salt thereof, has the following structure:

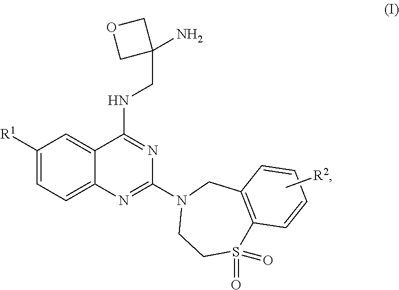

wherein

R¹ is selected from the group consisting of hydrogen, halogen, C₁-C₃ alkyl, C₁-C₃ alkoxyl, —CN, —C(O)R³, halogen substituted C₁-C₃ alkyl, and halogen substituted C₁-C₃ alkoxyl;

R² is selected from the group consisting of hydrogen, halogen, C₁-C₃ alkyl, C₁-C₃ alkoxyl, and —CN; and

R³ is selected from the group consisting of hydrogen, C₁-C₃ alkyl, and C₁-C₃ alkoxyl.

The compounds useful as described above can be formulated into pharmaceutical compositions for use in the treatment or prevention of RSV conditions. Standard pharmaceutical formulation techniques are used, such as those disclosed in Remington: The Science and Practice of Pharmacy, 21st Ed., Lippincott Williams & Wilkins (2005), incorporated herein by reference in its entirety.

In addition to the selected compounds useful as described above, some embodiments include, but are not limited to, compositions containing a pharmaceutically acceptable carrier.

The term “pharmaceutically acceptable carrier”, as used herein, means one or more compatible solid or liquid fillers, diluents or encapsulating substances, which are suitable for administration to a mammal. The term “compatible”, as used herein, means that the components of the composition are capable of being commingled with the subject compound, and with each other, in a manner such that there is no interaction, which would substantially reduce the pharmaceutical efficacy or increase the side effects of the composition under ordinary use situations. Pharmaceutically acceptable carriers must be of sufficiently high purity and sufficiently low toxicity to render them suitable for administration to a human subject being treated.

Some examples of substances, which can serve as pharmaceutically acceptable carriers or components thereof, include but are not limited to sugars, such as lactose, glucose, maltodextrin, and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid, sodium stearyl fumarate, and magnesium stearate; calcium sulfate; vegetable oils, such as peanut oil, cottonseed oil, sesame oil, olive oil, corn oil; polyols, such as propylene glycol, glycerin, sorbitol, mannitol, and polyethylene glycol; alginic acid; emulsifiers, such as the Tweens; wetting agents, such sodium lauryl sulfate; coloring agents; flavoring agents; tableting agents; stabilizers; antioxidants; preservatives; pyrogen-free water; isotonic saline; and phosphate buffer solutions.

The choice of a pharmaceutically acceptable carrier to be used in conjunction with the subject compound is basically determined by the way the compound is to be administered.

The compositions, as described herein, are preferably provided in unit dosage form. As used herein, a “unit dosage form” is a composition containing an amount of a compound that is suitable for administration to a human subject, in a single dose unit, according to good medical practice. The preparation of a single unit dosage form, however, does not imply that the dosage form is administered once per day or once per course of therapy. Such dosage forms are contemplated to be administered once, twice, thrice or more per day and multiple unit dosage forms may be administered at one time, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. Those skilled in the art will appreciate that oral and nasal compositions include compositions that are administered by inhalation, and are made using available methodologies. Depending upon the particular route of administration desired, a variety of pharmaceutically acceptable carriers well-known in the art may be used.

Pharmaceutically acceptable carriers include, but are not limited to, for example, solid or liquid fillers, diluents, hydrotropies, surface-active agents, and encapsulating substances. Optional pharmaceutically active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms, 8th Edition (2004), all incorporated herein by reference.

Various oral dosage forms can be used, including but not limited to such solid forms as tablets, capsules, granules, and bulk powders. Tablets can be compressed, triturated, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include, but are not limited to, aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents, and flavoring agents.

The pharmaceutically acceptable carriers suitable for the preparation of unit dosage forms for peroral administration is well-known in the art. Tablets typically comprise conventional pharmaceutically compatible adjuvants as inert diluents, such as calcium carbonate, sodium carbonate, mannitol, lactose, and cellulose; binders, such as starch, gelatin, and sucrose; disintegrants, such as starch, alginic acid, and croscarmellose; lubricants, such as magnesium stearate, stearic acid, and talc. Glidants, such as silicon dioxide, can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents useful as described above. The selection of carrier components depends on secondary considerations like taste, cost, and shelf stability, which are not critical, and can be readily made by a person skilled in the art.

Peroral compositions also include, but are not limited to, liquid solutions, emulsions, suspensions, and the like. The pharmaceutically acceptable carriers suitable for preparation of such compositions are well known in the art. Typical components of carriers for syrups, elixirs, emulsions and suspensions include, but are not limited to, ethanol, glycerol, propylene glycol, polyethylene glycol, liquid sucrose, sorbitol, and water. For a suspension, typical suspending agents include, but are not limited to, methyl cellulose, sodium carboxymethyl cellulose, AVICEL RC-591, tragacanth, and sodium alginate; typical wetting agents include, but are not limited to, lecithin and polysorbate 80; and typical preservatives include, but are not limited to, methyl paraben and sodium benzoate. Peroral liquid compositions may also contain one or more components such as sweeteners, flavoring agents, and colorants useful as described above.

Such compositions may also be coated by conventional methods, typically with pH or time-dependent coatings, such that the subject compound is released in the gastrointestinal tract in the vicinity of the desired topical application, or at various times to extend the desired action.

Such dosage forms typically include, but are not limited to, one or more of cellulose acetate phthalate, polyvinylacetate phthalate, hydroxypropyl methyl cellulose phthalate, ethyl cellulose, Eudragit coatings, waxes, and shellac.

The present invention provides optimized excipients for enteric coated micro pellets containing Compound (I) that are stable under acidic conditions and can be released inside animal and human body with a pharmacokinetic profile that provides sustained anti-RSV efficacy and reduced side effects.

In one embodiment, each enteric coated micro pellet comprises, preferably consists of, from the core to the outside, a core bead, an optional first sealing layer, a drug layer (or an API layer), an optional second sealing layer, and an enteric coating layer, with the mass increase of each layer being: 1-15% for the optional first sealing layer, 5-150% for the drug layer, 2-15% for the optional second sealing layer, and 5-25% for the enteric coating layer. The optional first sealing layer and the second sealing layer contains hydroxypropyl methyl cellulose or polyvinyl alcohol. The drug layer comprises, preferably consists of, the Compound (I) and adhesive(s). The enteric coating layer comprises, preferably consists of, enteric coating material(s), plasticizer(s), anti-caking agent(s), and emulsifier(s).

In another embodiment, each enteric coated micro pellet comprises, preferably consists of, 0.05-0.5 mg of the core bead, 0.001-0.01 mg of the optional first sealing layer, 0.01-0.1 mg of the drug layer, 0.002-0.02 mg of the optional second sealing layer, and 0.01-0.1 mg of the enteric coating layer.

The present invention also provides a dosing regimen for the administration of the composition at a therapeutically effective dosage, i.e., a dose and frequency sufficient to provide treatment or prevention for the RSV disease. While human dosage levels have yet to be optimized for the compounds of the preferred embodiments, generally, a daily dose for the preferred compounds as described herein is from about 100 mg to 600 mg per day for adults, and 1 mg to 10 mg per kilogram body weight per day for children. The amount and frequency of active compound administered will be dependent on the subject and disease state being treated, the severity of the affliction, the manner and schedule of the administration, and the judgment of the prescribing physician.

Oral administration of the compounds as described herein, or the pharmaceutically acceptable salts thereof, are customary in treating RSV disease that are the subject of the preferred embodiments.

In one embodiment, the pharmaceutical composition comprising capsules containing 30-1200 mg of Compound (I) in the form of enteric coated micro pellets was tested in a randomized, double-blind, placebo-controlled single and multiple ascending dose study in healthy adult subjects.

The following examples are for illustrative purposes only and are not intended, nor should they be construed as limiting the scope of the invention in any manner.

### Example 1: The Composition of Enteric Coated Micro Pellets

The composition of enteric coated micro pellets is shown below:

| Components | Weight (mass unit) | weight percentage (%) |
|---|---|---|
| Compound (I) | 100.0 | 22.86 |
| Sugar sphere | 200.0 | 45.71 |
| Opadry | 30.0 | 6.86 |
| HPMC | 40.0 | 9.14 |
| Eudragit L30D-55 | 50.0 | 11.43 |
| Triethyl citrate | 9.0 | 2.06 |
| Glycerin monostearate | 3.0 | 0.68 |
| Tween 80 | 2.5 | 0.57 |
| Silicon dioxide | 2.0 | 0.46 |
| Talc | 1.0 | 0.23 |
| Total amount | 437.5 | 100.00 |

### Example 2: The Composition of Enteric Coated Micro Pellets

The composition of enteric coated micro pellets is shown below:

| Components | Weight (mass unit) | weight percentage (%) |
|---|---|---|
| Compound (I) | 10.0 | 7.69 |
| Sugar sphere | 90.0 | 69.23 |
| HPMC | 5.0 | 3.85 |
| Eudragit L30D-55 | 7.0 | 5.38 |
| PlasACRYL ® HTP 20 | 5.0 | 3.85 |
| Opadry | 11.0 | 8.46 |
| Silicon dioxide | 1.0 | 0.77 |
| Talc | 1.0 | 0.77 |
| Total amount | 130.0 | 100.00 |

### Example 3: Stability and Dissolution Test

The enteric coated micro pellets containing Compound (I) was tested for stability in 0.1N HCl for 2 hours. Analysis of the test showed that only 0.2% of Compound (I) was released. Afterwards, the micro pellets were transferred to a pH=6.8 buffer solution containing 0.5 wt % sodium lauryl sulfate under standard dissolution test conditions. It was found that 93.1% of Compound (I) was released within 10 minutes, as shown in the following table.

| | Time (min) | | | | | | | | |
|---|---|---|---|---|---|---|---|---|---|
| | 0 | 120 | 125 | 130 | 135 | 150 | 165 | 180 | 210 |
| Release (%) | 0.0 | 0.2 | 55.7 | 93.1 | 97.8 | 98.0 | 98.4 | 98.7 | 98.8 |

| | | |
|---|---|---|
| Medium | 0.1N HCl | pH = 6.8 buffer solution |

### Example 4: Pharmacokinetic Studies Comparing Suspension Formulation and Enteric Coated Micro Pellet Formulation of Compound (I) in Dogs

The animals were administered suspension formulation or enteric coated micro pellet formulation of Compound (I) at 10 mg/kg dose. The plasma concentration was monitored over a 24-hour period. The result demonstrated that the total exposure of Compound (I) was comparable between the two formulations. However, the maximum concentration for the enteric coated micro pellet formulation was significantly lower than the suspension formulation while the drug concentration at 12 hours after dosing is significantly higher for the micro pellets.

These differential pharmacokinetic properties are desirable for extended drug action and reduced side effects, and thus, a wider drug safety window and a higher anti-RSV efficacy.

| | Dose | Tₘₐₓ | Cₘₐₓ | C_{12 h} | AUC_{0-inf} |
|---|---|---|---|---|---|
| Formulation | (mg/kg) | (h) | (ng/ml) | (ng/ml) | (ng · h/ml) |
| Suspension | 10 | 0.83 | 282 | 10.4 | 1220 |
| Micro pellets | 10 | 2.33 | 102 | 20.4 | 800 |

Tₘₐₓ: time to reach maximum drug concentration;
Cₘₐₓ: maximum drug concentration;
C_{12 h}: drug concentration at 12 hours after dosing;
AUC_{0-inf}: area under the drug concentration-time curve from time 0 to infinity.

### Example 5: A Randomized, Double-Blind, Placebo-Controlled Single and Multiple Ascending Dose Study Using Enteric Coated Micro Pellets in Healthy Adult Subjects

Capsules of enteric coated micro pellets containing Compound (I) were administered to healthy adult volunteers as a single dose at increasing dose levels as well as multiple doses (twice daily for 7 days) at three different dose levels. The number of subjects exposed to Compound (I) are summarized in the table below.

| Treatment arm | Dose (mg) | No. of subjects |
|---|---|---|
| Single dose | 30 | 6 |
| | 100 | 6 |
| | 300 | 6 |
| | 600 | 8 |
| | 1200 | 6 |
| Multiple dose | 100 | 6 |
| (BID for 7 days) | 200 | 6 |
| | 300 | 6 |

Pharmacokinetic data following a single dose indicated that Compound (I) is well absorbed with median Tₘₐₓ ranging between 2.0 h and 3.5 h across the range of 30 mg - 1200 mg. Exposure, in terms of Cₘₐₓ and AUC₀₋ₜ, increases with increasing doses of Compound (I) up to 300 mg. However, non-proportionality was observed between 600 and 1200 mg. Estimates of Tₘₐₓ appear to remain consistent across dose levels, and ranges between 2.0 h and 3.5 h. Drug elimination half-life, t½, was also comparable across the dose groups above 30 mg, ranging from ˜6-12 hours. These findings suggest first-order, linear kinetics in humans for the micro pellets.

Human PK profile of the micro pellet formulation of Compound (I) at dose levels from 30 mg to 1200 mg in the single ascending dose treatment arm is summarized in the table below.

| Dose Level | AUC₀₋ₜ | AUC_{0-inf} | Cₘₐₓ | Tₘₐₓ | t_{1/2} |
|---|---|---|---|---|---|
| (single dose) | (ng · h/mL) | (ng · h/mL) | (ng/mL) | (h) | (h) |
| 30 mg | 238 | 286 | 42 | 3.50 | 5.92 |
| 100 mg | 1310 | 1940 | 181 | 2.00 | 7.42 |
| 300 mg | 2770 | 5420 | 404 | 2.00 | 11.90 |
| 600 mg | 7290 | 7490 | 333 | 3.50 | 7.42 |
| 1200 mg | 7340 | 8640 | 596 | 3.00 | 10.50 |

In the multiple ascending dose treatment arm, following repeated twice-daily dosing for six and a half days, results at steady state on Day 7 revealed no marked differences amongst any of the parameters compared across cohorts in terms of dose-normalized AUC₀₋ₜ, AUC_{0-inf}, and Cₘₐₓ. Tₘₐₓ also does not appear to be affected by repeated dose administration over the period with results very similar to that observed in the single dose arm. With respect to elimination, t½, values were comparable for all dose groups with median estimates of t, similar to that of the single dose arm, and appears to be independent of dose. The table below summarizes the PK profile of the micro pellet formulation of Compound (I) at dose level from 100 mg to 300 mg in the multiple ascending dose treatment arm.

| Dose Level | AUC₀₋ₜ | AUC_{0-inf} | Cₘₐₓ | Tₘₐₓ | t_{1/2} |
|---|---|---|---|---|---|
| (multiple dose) | (ng · h/mL) | (ng · h/mL) | (ng/mL) | (h) | (h) |
| 100 mg | 2240 | 3590 | 177 | 2.50 | 12.7 |
| 200 mg | 4360 | 4690 | 381 | 2.50 | 10.7 |
| 300 mg | 4740 | 6340 | 483 | 3.00 | 9.55 |

### Example 6: An Open Label, Single-Dose Study Using Compound (I) as a Solution in Healthy Adult Subjects

In this study, Compound (I) was administered to healthy male volunteers as a solution in aqueous tartaric acid. The number of subjects and exposure to Compound (I) are summarized in the table below.

| | Dose level | No. of |
|---|---|---|
| Formulation | (single dose) | subjects |
| Solution | 300 mg | 7 |

The pharmacokinetic results showed that Compound (I) was rapidly absorbed with Tₘₐₓ values between 0.5 and 1.0 h, significantly shorter than that of the enteric micro pellets. The drug concentration declined with a mean geometric half-life of 10.3 h, similar to the micro pellets. Exposure in terms of Cₘₐₓ reached 4-5 folds of that observed with micro pellets, while the AUC were similar at the same dose level. The human PK parameters of the solution formulation was summarized in the table below.

| | AUC₀₋ₜ | AUC_{0-inf} | Cₘₐₓ | Tₘₐₓ | t_{1/2} |
|---|---|---|---|---|---|
| Dose Level | (ng · h/mL) | (ng · h/mL) | (ng/mL) | (h) | (h) |
| 300 mg | 6350 | 6380 | 2020 | 0.5-1.0 | 10.3 |

Although the present invention has been fully described in connection with the embodients thereof, it will be apparent to those skilled in the art that various modifications and variations can be made without departing from the spirit or scope of the present invention. Thus, it is intended that such modifications and variations are to be understood as being included within the scope of the present invention as defined by the appended claims and their equivalents.

1. A pharmaceutical unit dosage composition comprising a plurality of enteric coated micro pellets, each enteric coated micro pellet comprising
a core bead;
an optional first sealing layer;
an active pharmaceutical ingredient (API) layer, the API layer including a Compound (I) having the following structure or a pharmaceutically acceptable salt thereof, 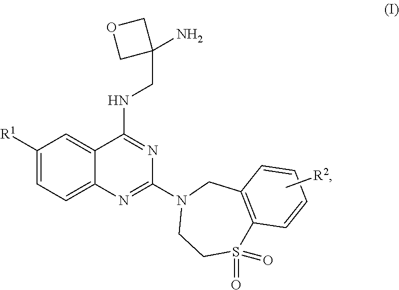
wherein
R¹ is selected from the group consisting of hydrogen, halogen, C₁-C₃ alkyl, C₁-C₃ alkoxyl, —CN, —C(O)R³, halogen substituted C₁-C₃ alkyl, and halogen substituted C₁-C₃ alkoxyl;
R² is selected from the group consisting of hydrogen, halogen, C₁-C₃ alkyl, C₁-C₃ alkoxyl, and —CN; and
R³ is selected from the group consisting of hydrogen, C₁-C₃ alkyl, and C₁-C₃ alkoxyl;
an optional second sealing layer; and
an enteric coating layer.

2. The pharmaceutical unit dosage composition according to claim 1, wherein
in the structure of the Compound (I), R¹ is methyl and R² is hydrogen.

3. The pharmaceutical unit dosage composition according to claim 1, wherein
the pharmaceutical unit dosage composition includes 10 to 300 mg of the Compound (I).

4. The pharmaceutical unit dosage composition according to claim 1, wherein
the pharmaceutical unit dosage composition is in a form selected from the group consisting of a capsule, a tablet, and a sachet.

5. The pharmaceutical unit dosage composition according to claim 1, wherein
the core bead is selected from the group consisting of a sucrose sphere, a microcrystalline cellulose sphere, and a starch sphere;
the core bead has a diameter of 0.2 to 2 mm; and
the weight of the core bead is 0.05 to 0.5 mg.

6. The pharmaceutical unit dosage composition according to claim 1, wherein
the optional first sealing layer includes an adhesive agent;
the adhesive agent is selected from the group consisting of hydroxypropyl methyl cellulose, hydroxyethyl cellulose, hydroxypropyl cellulose, sodium carboxymethyl cellulose, polyvinyl alcohol, polyvinyl pyrrolidone, starch slurry, methylcellulose, and a combination thereof; and
the weight of the optional first sealing layer is 0.001 to 0.01 mg.

7. The pharmaceutical unit dosage composition according to claim 6, wherein
the optional first sealing layer also includes talc.

8. The pharmaceutical unit dosage composition according to claim 1, wherein
the optional first sealing layer is made by using gastric soluble film coating premix; and
the weight of the optional first sealing layer is 0.001 to 0.01 mg.

9. The pharmaceutical unit dosage composition according to claim 1, wherein
the API layer includes the Compound (I) and an adhesive agent;
the adhesive agent is selected from the group consisting of hydroxypropyl methyl cellulose, hydroxyethyl cellulose, hydroxypropyl cellulose, polyvinyl alcohol, sodium carboxymethyl cellulose, polyvinyl pyrrolidone, starch slurry, methylcellulose, and a combination thereof, and
the weight of the API layer is 0.01 to 0.1 mg.

10. The pharmaceutical unit dosage composition according to claim 1, wherein
the optional second sealing layer includes an adhesive agent;
the adhesive agent is selected from the group consisting of hydroxypropyl methyl cellulose, hydroxyethyl cellulose, hydroxypropyl cellulose, sodium carboxymethyl cellulose, polyvinyl alcohol, polyvinyl pyrrolidone, starch slurry, methylcellulose, ethylcellulose, and a combination thereof, and
the weight of the optional second sealing layer is 0.002 to 0.02 mg.

11. The pharmaceutical unit dosage composition according to claim 10, wherein
the optional second sealing layer also includes talc.

12. The pharmaceutical unit dosage composition according to claim 1, wherein
the optional second sealing layer is made by using gastric soluble film coating premix; and
the weight of the optional second sealing layer is 0.002 to 0.02 mg.

13. The pharmaceutical unit dosage composition according to claim 1, wherein
the enteric coating layer includes 30-95 wt % of an enteric coating material, 1-40 wt % of a plasticizer, 1-20 wt % of an anti-caking agent, and 0.5-20 wt % of an emulsifier;
the enteric coating material is selected from the group consisting of acrylic resin, hydroxypropyl methyl cellulose, hydroxypropyl methyl cellulose phthalate, cellulose acetate phthalate, polymethacrylates, methacrylic acid-ethyl acrylate copolymer, methacrylic acid copolymer, ethylene-vinyl acetate copolymer, and a combination thereof,
the plasticizer is selected from the group consisting of triethyl citrate, polyethylene glycol, tributyl citrate, dibutyl sebacate, diethyl phthalate, and a combination thereof;
the anti-caking agent is selected from the group consisting of glycerin monostearate and talc;
the emulsifier is selected from the group consisting of Tween and sodium lauryl sulfate; and
the weight of the enteric coating layer is 0.01 to 0.1 mg.

14. The pharmaceutical unit dosage composition according to claim 1, wherein
the enteric coating layer includes at least one of Eudragit L30D-55, Eudragit S100, and Eudragit L100; and
the weight of the enteric coating layer is 0.01 to 0.1 mg.

15. The pharmaceutical unit dosage composition according to claim 1, wherein
the enteric coating layer includes mixed emulsion of plasticizer; and
the weight of the enteric coating layer is 0.01 to 0.1 mg.

16. The pharmaceutical unit dosage composition according to claim 1, wherein
the pharmaceutical unit dosage composition includes a plurality of enteric coated micro pellets and an anti-sticking agent;
the anti-sticking agent is selected from the group consisting of silicon dioxide, stearic acid, sodium stearyl fumarate, magnesium stearate, talc, and a combination thereof; and
the weight ratio of the anti-sticking agent to the enteric coated micro pellets is 0.0005-0.1:1.

17. The pharmaceutical unit dosage composition according to claim 1, wherein
the particle size D₉₀ of the Compound (I) is no more than 100 m.

18. A method for the treatment and prophylaxis of RSV diseases comprising
providing a Compound (I) having the following structure or a pharmaceutically acceptable salt thereof, 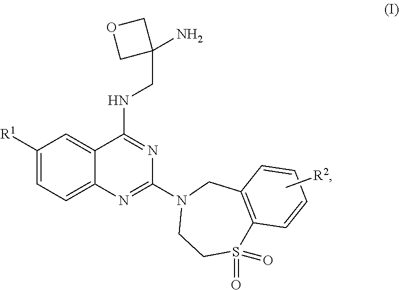
wherein
R¹ is selected from the group consisting of hydrogen, halogen, C₁-C₃ alkyl, C₁-C₃ alkoxyl, —CN, —C(O)R³, halogen substituted C₁-C₃ alkyl, and halogen substituted C₁-C₃ alkoxyl;
R² is selected from the group consisting of hydrogen, halogen, C₁-C₃ alkyl, C₁-C₃ alkoxyl, and —CN; and
R³ is selected from the group consisting of hydrogen, C₁-C₃ alkyl, and C₁-C₃ alkoxyl; and
administering to a patient in need thereof a therapeutically effective amount of the Compound (I).

19. The method according to claim 18, wherein
in the structure of the Compound (I), R¹ is methyl and R² is hydrogen.

20. The method according to claim 18, wherein
the therapeutically effective amount of the Compound (I) is 200 mg to 600 mg once per day for adult patients.

21. The method according to claim 18, wherein
the therapeutically effective amount of the Compound (I) is 100 mg to 300 mg every 12 hours for adult patients.

22. The method according to claim 18, wherein
the therapeutically effective amount of the Compound (I) is 1 mg to 10 mg per kilogram body weight once per day for pediatric patients.

23. The method according to claim 18, wherein
the therapeutically effective amount of the Compound (I) is 1 mg to 8 mg per kilogram body weight every 12 hours for pediatric patients.

24. The method according to claim 18, wherein
when administering to a patient in need thereof a therapeutically effective amount of the Compound (I), an elimination half-life (t½) of the Compound (I) is about 6 to 13 hours.
